(19) United States
(12) Patent Application Publication
(10) Pub. No.: US 2016/0051295 A1
(43) Pub. Date: Feb. 25, 2016

(54) OSTEOSYNTHESIS DEVICE

(71) Applicants: Kurume University, Kurume-shi, Fukuoka (JP); K. N. MEDICAL. CO., LTD., Fukuoka-shi, Fukuoka (JP)

(72) Inventors: Hidetomo NAKAMURA, Fukuoka-shi, Fukuoka (JP); Ryuta FUKUTOYO, Fukuoka-shi, Fukuoka (JP)

(73) Assignees: K. N. MEDICAL. CO., LTD., Fukuoka-shi, Fukuoka (JP); Kurume University, Kurume-shi, Fukuoka (JP)

(21) Appl. No.: 14/783,198

(22) PCT Filed: Apr. 9, 2014
(86) PCT No.: PCT/JP2014/060328

§ 371 Date: Oct. 8, 2015

(30) Foreign Application Priority Data
Apr. 11, 2013 (JP) 2013-082661

(51) Int. Cl. A61B 17/72; A61B 17/80

(52) U.S. Cl. CPC A61B 17/7225; A61B 17/809

(57) ABSTRACT

An osteosynthesis device is provided, which, while being a single device, is wide in a range of fixing positions or fixing methods that can be selected in a repositioning operation, enables firm fixation of an affected portion, and alleviates feeling of discomfort felt at the affected portion after the operation.

An osteosynthesis device D1 is a device used, in case of fracture of an olecranon side of an ulna, to fix and reposition respective bone fragments and includes an intramedullary nail 1 inserted into the ulna 3, an end plate 2a mounted on an outer surface of the ulna at the olecranon 31 side, and a threaded screw N1 arranged to screw on the end plate to the intramedullary nail. Screws S are used in combination with the osteosynthesis device D1 to fix, etc., the inserted intramedullary nail 1 onto the ulna 3. The intramedullary nail 1, which is a round rod body having a tapering tip end portion and has a thickness enabling entry into the marrow of the ulna and a predetermined length, includes a threaded hole 111 formed in a base end surface 101, long holes 114 and 115 formed at a base end side with respect to a central portion of a circumferential surface 102, and circular holes 112, 113, 116, 117, and 118 formed at predetermined parts between a tip portion side and the base end side of the circumferential surface 102.

### CROSS REFERENCE TO RELATED APPLICATIONS

This patent application is a U.S. national stage application under 35 U.S.C. §371 of International Patent Application No. PCT/JP2014/060328 filed on Apr. 9, 2014, which claims the benefit of foreign priority to Japanese Patent Application No. JP 2013-082661 filed on Apr. 11, 2013. The International application was published in Japanese on Oct. 16, 2014, as International Publication No. WO 2014/168184 A1 under PCT Article 21(2).

### TECHNICAL FIELD

The present invention relates to an osteosynthesis device. More specifically, it relates to an osteosynthesis device used, in case of fracture of an olecranon side of an ulna, to fix and reposition respective bone portions to the original bone shape.

### BACKGROUND ART

There is a surprisingly high number of cases of fracture of an olecranon side of an ulna and although for treatment thereof, there are the so-called “plate fixation method (an operation method using a curved plate and fixing threaded screws therefor where the plate is positioned so as to be aligned along the shape of repositioned bone fragments and mounted to the ulna by the fixing threaded screws)” and the “tension band wiring method (an operation method using two inserted wires and a binding wire made of stainless steel where, after setting the bone fragments back in the original state, the two inserted wires are inserted in parallel into a marrow to fix the bone fragments and the binding wire is passed through a side hole bored in a bone fragment corresponding to tip portion sides of the inserted wires, crossed once at an intermediate portion, wound around base end portions of both inserted wires that are outside the bone fragment, and has its two end portions twisted tightly together to achieve fixation),” these methods have several drawbacks.

For example, with the plate fixation method, a metal plate is set along a wide range of an elbow portion, causing a patient to feel extreme discomfort. Also with the tension band wiring method, there has occurred a problem where despite the nails inserted into the marrow being fastened and fixed by the wire, the fastening, by the wire, of the nails installed inside the marrow loosens with the elapse of time after operation. The present inventors have proposed the osteosynthesis device described in Patent Document 1 as a device that is lower in physical burden to a patient, does not inflict discomfort, is further installed and held with stability for a long period of time without loosening, and is used in an operation method by which satisfactory convalescence of an affected portion is achieved.

The osteosynthesis device according to Patent Document 1 includes an intramedullary nail having a predetermined length and a thickness enabling passage inside a marrow of an ulna, a fixing washer having a predetermined number of sharp elements that can be erected on an outer surface of an olecranon side of the ulna, a base portion fixing threaded screw, and tip portion fixing threaded screws respectively of predetermined numbers, the intramedullary nail having formed therein a threaded hole, provided in a length direction from a base end portion and enabling the base portion fixing threaded screw to be screwed thereinto, and a tip portion fixing threaded screw insertion hole, provided at a tip portion side and penetrating through in a direction intersecting the length direction, and the fixing washer having formed therein a base portion fixing threaded screw insertion hole, through which the base portion fixing threaded screw is passed.

The osteosynthesis device exhibits the following actions and effects.

(a) A central bone fragment of an ulnar end portion is held by fastening of the fixing washer (may also be referred to as the “end plate”; the same applies hereinafter) with a large portion being fixed by the sharp elements so that forces are dispersed, making the central bone fragment unlikely to break and further, the respective bone fragments can be integrated so as to be joined in the length direction of the intramedullary nail by the fastening of the fixing washer. There is thus hardly any portion that is exposed on the surface of the ulna besides the fixing washer after operation and feeling of discomfort by an operated patient him/herself after the operation can thus be prevented or alleviated.

Also, the portion at which skin is cut in the operation is made smaller than in the conventional case, alleviating the danger of infection and reducing the physical burden on the part of the patient. Further, the intramedullary nail is held by the end plate and the associated threaded screw so that loosening, which is a disadvantage of the tension band wiring method, does not occur.

(b) By performing operation using the osteosynthesis device, the device is integrated firmly with the ulna, making the form of the osteosynthesis device unlikely to change after the operation and thereby enabling prevention of deformation of an articular surface shape of a trochlear notch of the ulna due to change of form of the device as well as prevention of arising of a restriction different from that which is inherent within a movable range of the elbow joint.

### CITATION LIST

### Patent Document

Patent Document 1: International Publication No. 2010/005076

### SUMMARY OF INVENTION

### Technical Problem

Although the osteosynthesis device of Patent Document 1 exhibits the actions and effects described above, is useful in the process of operation, and provides excellent effects in regard to osteosynthesis and feeling of installation in comparison to the conventional osteosynthesis devices, upon further examination of the arrangement of the osteosynthesis device, it is postulated that a patient after operation may still feel discomfort with the surface shape of the fixing washer at the portion of installation at the olecranon and with the projections of the threaded screw heads of the tip portion fixing threaded screws at a forearm portion.

Also, although operation methods for a fracture of an elbow portion include, for example, a method where the fractured portions are fitted together completely and made to undergo regenerative healing, a method where interval adjustment by forming of a gap between fractured portions, etc., is performed without performing complete fitting to make the bone undergo regenerative healing to its original length, etc., and the operation method may differ according to the treating physician, it was difficult to accommodate individually for such different operation methods with the osteosynthesis device of Patent Document 1.

Further, for a fracture of multiple parts in a vicinity of the elbow portion, there is an operation method where, in order to reposition the parts, a small bone fragment is joined to a large bone fragment or small bone fragments are joined to each other by a fixing means, such as a fixing pin or screw, etc., and in performing such operation methods, the present inventors arrived at an idea that, in performing such an operation method, by combining the osteosynthesis device according to Patent Document 1 with a plurality or a plurality of types of fixing means to perform the operation, a firmer fixation is made possible, thereby contributing to improvement of the reliability of fixation.

### Object of the Invention

An object of the invention is thus to provide an osteosynthesis device, which, while being a single device, is arranged to be wide in a selection range of fixing positions or fixing methods that an operator can implement in a repositioning operation and yet enable firm fixation of an affected portion and further enable alleviation of feeling of discomfort that a patient feels at the affected portion after the operation.

### Solution to Problem

The means according to the present invention made to achieve the above object are as follows.

(1) The present invention is an osteosynthesis device that includes an intramedullary nail, which, when an olecranon side of an ulna is fractured, is used by being inserted into the ulna to fix and reposition respective bone fragments, the intramedullary nail having a threaded hole, formed in a base end surface, a long hole, formed in a circumferential surface such that both opening portions of the long hole are the same in aperture and a fixing pin or screw can be inserted therethrough, and at least two or more circular holes, each formed in the circumferential surface and enabling a fixing pin or screw to be inserted therethrough, the long hole being provided at a part somewhere between a center and a base end in a length direction of the intramedullary nail, with a major axis of the long hole being the same in direction as the length direction of the intramedullary nail, and being arranged so that an insertion angle of the fixing pin or screw inserted into the long hole may be selected freely from among any angle orthogonal to the length direction of the intramedullary nail, inclined in a direction from a tip portion direction to a base portion direction of the intramedullary nail, or inclined in a direction from the base end direction to the tip portion direction of the intramedullary nail, and the circular holes being formed at parts somewhere between a tip end and the base end in the length direction of the intramedullary nail with formation directions of the circular holes being orthogonal, inclined, or a combination thereof with respect to the length direction of the intramedullary nail and being such that, with respect to a diameter direction of the intramedullary nail, the respective holes are equally overlapped with the diameter direction or a portion or all of the holes intersect the diameter direction at arbitrary angles.

The invention of (1) above is used in repositioning operation of an ulna that is fractured at one or two or more parts (including not only cases of complete fracture but also cases of incomplete fracture and compound fracture; the same applies hereinafter). An operator cuts the skin at the olecranon, drills a hole in the bone at the corresponding part, and inserts the intramedullary nail from the drilled portion into the bone marrow (this shall be abbreviated hereinafter as “inserts into the ulna”).

The operator then pierces a fixing pin or screw through a hole, among the circular holes and the long hole of the inserted intramedullary nail, that is positioned at a portion suited to the operator's operation method to fix the intramedullary nail to the ulna and fix the fractured parts (in the case of a compound fracture, etc., bone fragments that are separated, detached, or free from the ulna, including those present on an extension of the pierced fixing pin or screw; the same applies hereinafter).

Further, with the long hole, the operator can pierce the fixing pin or screw at any angle with respect to the major axis direction of the hole (for example, obliquely with respect to the major axis direction of the hole). That is, the operator pierces the fixing pin or screw in a direction suited to the operator's operating method to fix the intramedullary nail to the ulna and fix the bone fragments.

In the present specification and claims, to “pierce” a fixing pin or screw refers to both cases of piercing simply by thrusting through and cases of piercing a fixing pin or screw while rotating it.

(2) The invention may include an end plate mounted on an outer surface of an elbow side of the ulna and a threaded screw by which the end plate is screwed onto a threaded hole in a base end surface of the intramedullary nail, the end plate may have a base plate with one or more penetrating holes formed in a thickness direction and a plurality of projections projecting from the base plate toward the ulna side and contacting an outer surface of the olecranon side of the ulna, a surface of the base plate at the side opposite the ulna may be formed to have no projections or to be smooth, one of either or both of the base plate and the projections of the end plate may have flexibility or elasticity, the endplate may be arranged to press the olecranon toward a tip portion side of the ulna by means of an elastic force of the base plate or the projections, and the threaded screw may be used by being inserted in any one of the penetrating holes formed in the end plate.

With the invention of (2) above, when an operator, who has selected the use of the intramedullary nail and the end plate, makes the respective projections of the end plate contact the outer surface of the elbow side of the ulna and screws the end plate onto the intramedullary nail by means of the threaded screw, the end plate, by virtue of the elastic force that it provides, presses the olecranon of the ulna toward the tip portion side of the ulna (it may also be said that the end plate is drawn in the direction of the intramedullary nail inside the ulna; the same applies hereinafter). The use of the threaded screw facilitates adjustment of the force by which the end plate presses the olecranon of the ulna in the mounting process or adjustment in forming an interval (gap) between fractured portions.

The base plate front surface (the surface at the side opposite the ulna; the same applies hereinafter) of the mounted end plate has no projections or is smooth and therefore discomfort felt by a patient at the corresponding location after operation, possibility of occurrence of a pressure sore, and influence on tendons, etc., in the vicinity are alleviated.

(3) The present invention is an osteosynthesis device, which, when an olecranon side of an ulna is fractured, is used to fix and reposition respective bone fragments and includes an intramedullary nail inserted into the ulna, a threaded screw screwed on along a length direction of the intramedullary nail from a base end of the intramedullary nail, and a fixing pin or screw fixing the inserted intramedullary nail onto the ulna, the intramedullary nail having a threaded hole formed in a base end surface, at least one or more tip portion side holes, each being a hole provided in a circumferential surface within a range of ⅓rd the length from a tip end portion in the length direction, and at least two or more base portion side holes, each being a hole through which the fixing pin or screw is inserted and which is provided in the circumferential surface within a range from a central portion to a base end portion in the length direction, the holes being provided so that, with respect to a cracked portion or fractured portion of the ulna, a plurality of the base portion side holes are positioned across the cracked portion or fractured portion or a base portion side hole and a tip portion side hole are positioned so as to sandwich the cracked portion or fractured portion, and the base portion side holes and the tip portion side holes forming a positional relationship of respective holes with which insertion angles of the fixing pin or screw are combined upon being freely selected from among angles orthogonal and inclined with respect to the length direction of the intramedullary nail and angles such that, when viewed from the base end portion to the tip end portion, the respective holes are overlapped equally or intersect at arbitrary angles.

The intramedullary nail according to the invention of (3) above is inserted into the ulna by the same procedures as the invention of (1) above. The operator then pierces the fixing pin or screw through a hole, among the base portion side holes and the tip portion side holes of the inserted intramedullary nail, that is positioned at a portion suited to the operator's operation method to fix the intramedullary nail to the ulna and fix the fractured parts.

(4) The present invention may further include, in the osteosynthesis device, an end plate mounted on an outer surface of an elbow side of the ulna, the end plate may have a penetrating hole, through which is inserted the threaded screw that is inserted and screwed on along the length direction of the intramedullary nail from the base end of the intramedullary nail, and the threaded screw may be inserted and screwed on through the end plate.

With the invention of (4) above, the operator, who has selected the use of the intramedullary nail and the end plate, makes the end plate contact the outer surface of the elbow side of the ulna and screws the end plate onto the intramedullary nail by means of the threaded screw inserted through the penetrating hole.

(5) The present invention may be such that the end plate further has one or more penetrating holes formed in a thickness direction of a base plate of the end plate and a plurality of projections projecting from the base plate toward the ulna side and contacting an outer surface of the olecranon side of the ulna, each added penetrating hole being a hole through which is inserted a fixing pin or screw that is used together with the projections to fix the intramedullary nail near the elbow.

With the invention of (5) above, the operator makes the respective projections of the end plate contact the outer surface of the elbow side of the ulna and screws the end plate onto the intramedullary nail by means of the threaded screw. Further, the operator inserts and passes the fixing pin or screw through the added penetrating hole as necessary and pierces the fixing pin or screw into the olecranon of the ulna or a vicinity portion thereof to fortify the fixation of the fractured parts near the elbow.

(6) The invention of (2) above may be such that the end plate further has one or more penetrating holes formed in the thickness direction of the base plate, each added penetrating hole being a hole through which is inserted a fixing pin or screw that is used to fix the intramedullary nail near the elbow.

With the invention of (6) above, the operator inserts and passes the fixing pin or screw through the added penetrating hole as necessary and pierces the fixing pin or screw into the olecranon of the ulna or a portion in the vicinity thereof to fortify the fixation of the fractured parts near the elbow.

(7) The invention of (5) above may be such that one of either or both of the base plate and the projections of the endplate has or have flexibility or elasticity, the endplate being arranged to press the olecranon toward a tip portion side of the ulna by means of the elastic force of the base plate or the projections and a surface of the base plate at the side opposite the ulna being formed to have no projections or to be smooth.

With the invention of (7) above, when the operator makes the respective projections of the end plate contact the outer surface of the elbow side of the ulna and screws the end plate onto the intramedullary nail by means of the threaded screw, the end plate, by virtue of the elastic force that it provides, presses the olecranon of the ulna toward the tip portion side of the ulna. The use of the threaded screw facilitates adjustment of the force by which the end plate presses the olecranon of the ulna in the mounting process or adjustment in forming an interval (gap) between fractured portions. The base plate front surface of the mounted end plate has no projections or is smooth and therefore the discomfort felt by a patient at the corresponding location after operation, the possibility of occurrence of a pressure sore, and the influence on tendons, etc., in the vicinity are alleviated.

(8) The invention may be such that the end plate is arranged with a peripheral edge portion of each of all or a portion of the penetrating holes formed in the base plate being recessed further than the base plate front surface so that a head portion of an installed threaded screw, fixing pin, or screw fits within the recess of the peripheral edge portion and a top surface of the head portion does not protrude above the base plate front surface or the base plate front surface is flush with the head portion.

With the invention of (8) above, the head portion top surface of the fixing pin or screw does not protrude from the base plate front surface of the endplate or the base plate front surface is flush with the head portion so that the base plate front surface of the end plate has no projections or is smooth and therefore the discomfort felt by a patient at the corresponding location after operation, the possibility of occurrence of a pressure sore, and the influence on tendons, etc., in the vicinity are alleviated.

(9) The invention may be such that a shape of the base end surface of the intramedullary nail is formed to a shape conforming to an olecranon shape that includes a curved shape or formed to an obliquely-cut planar or curved shape.

With the invention of (9) above, the base end surface of the intramedullary nail has the shape conforming to the olecranon portion shape, etc., and therefore even if the operator selects an operation method using the intramedullary nail alone (without using the end plate), the inserted intramedullary nail does not protrude from the olecranon side of the ulna or is unlikely to form a protrusion.

(10) The invention of (9) above may be such that in the case where the shape of the base end surface of the intramedullary nail is obliquely cut, the shape is a planar shape that is cut in an angular range from 5° to 30°.

With the invention of (10) above, the inserted intramedullary nail is more unlikely to protrude from the olecranon side of the ulna or is more unlikely to form a protrusion.

(11) The invention of (3) or (4) above may be such that at least two of the base portion side holes are positioned near a coronoid process of the ulna and, among the holes positioned near the coronoid process, the hole that is close to the base end portion is inclined from the base end direction to the tip end direction of the intramedullary nail so that the hole is directed in a tip end direction of the coronoid process, and the hole that is close to the tip end portion is inclined from the tip end direction to the base end direction of the intramedullary nail so that the hole is directed in the tip end direction of the coronoid process.

With the invention of (11) above, the respective base portion side holes positioned near the coronoid process of the ulna are inclined in the direction mentioned above and therefore the tip portion of a fixing pin or screw that is pierced through the hole is guided toward the tip end direction of the coronoid process.

(12) The invention may be such that at least one or more of the base portion side holes is a hole with a major axis and opening long in the same direction as the length direction of the intramedullary nail.

With the invention of (12) above, the operator can pierce a fixing pin or screw through a hole, among the holes of the inserted intramedullary nail, that is positioned at a portion suited to the operator's operation method and further pierce the fixing pin or screw at any angle with respect to the major axis direction of the hole (for example, obliquely with respect to the major axis direction of the hole). That is, the operator pierces the fixing pin or screw in a direction suited to the operator's operating method to fix the intramedullary nail to the ulna and fix the bone fragments.

(13) The invention may be such that at least one of the holes with a major axis and opening long in the same direction as the length direction of the intramedullary nail is positioned further to the base end side than a fractured or cracked portion.

With the invention of (13) above, the fractured or cracked portion is fixed by a fixing pin or screw that is pierced from the base end side toward the tip end side through the hole.

(14) The invention may be such that two holes positioned near the coronoid process are holes each with a major axis and opening long in the same direction as the length direction of the intramedullary nail.

With the invention of (14) above, in regard to a fixing pin or screw used in each of the holes, the fixing pin or screw can be pierced at any angle with respect to the major axis direction of the hole that is the length direction of the intramedullary nail (for example, obliquely with respect to the major axis direction of the hole). That is, the operator pierces the fixing pin or screw in a direction suited to the operator's operating method to fix the intramedullary nail to the ulna (in particular, the coronoid process) and fix the bone fragments.

(15) The invention may be such that a fixing pin or screw used in a hole, positioned near the coronoid process and through which the fixing pin or screw is inserted, has a shaft length reaching a base end or a tip end portion of the coronoid process of the ulna through the hole and capable of fastening together respective bone fragments that are separated or have a crack formed thereamong.

With the invention of (15) above, the fixation of the fractured portions is fortified and the range of selection of operation method is broadened because the fixing pin or screw that is pierced through a hole (that may be the long hole) positioned near the coronoid process reaches the base end or the tip end portion of the coronoid process and is further capable of fastening together the respective bone fragments that are separated or have a crack formed thereamong.

(16) The invention may be such that the intramedullary nail is a cylindrical body having a rectilinear hole extending from the base end to the tip end or has a rectilinear insertion hole extending from the base end of the intramedullary nail to the interior of a long hole positioned at a tip portion side or the interior of a hole with a major axis and opening long in the same direction as the length direction of the intramedullary nail.

With the invention of (16) above, the operator can select an operation method where a fixing pin or screw is pierced into the long hole or the hole with the major axis and opening long in the same direction as the length direction of the intramedullary nail, that is provided in the intramedullary nail to fix the intramedullary nail to the ulna and a rod-like object is inserted from the base end of the intramedullary nail into the interior of the hole or insertion hole to slide the fixed fixing pin or screw in the major axis direction of the same hole to move the fixed ulna to adjust an interval between fractured portions.

(17) The invention may be such that a female thread is provided on an inner circumferential surface of a hole, which is formed in the intramedullary nail and through which a fixing pin or screw is inserted, or an inner circumferential surface of a hole, which is formed in the end plate and through which a fixing pin or screw is inserted.

With the invention of (17) above, when an operation using a screw is performed, the screw will be in threaded engagement with the female thread on the hole wall of each circular hole of the intramedullary nail after the operation and will practically not fall out naturally.

(18) The invention may be such that a head portion or a base end of a fixing pin or screw is formed so as not to protrude further than an insertion portion in a circumferential surface of the ulna.

With the invention of (18) above, the base end portion of the pierced fixing pin or screw does not project or project greatly further than the circumferential surface of the ulna and therefore the discomfort felt by a patient at the corresponding pierced location after operation, the possibility of occurrence of a pressure sore, and the influence on tendons, etc., in the vicinity are alleviated.

(19) The invention may be such that at least the circular hole or the tip portion side hole that is positioned most distally is such that the formation angle of the circular hole, with a vertical direction of the intramedullary nail diameter being 0°, is within a range from 10° to 35°.

With the invention of (19) above, a process of piercing a fixing pin or screw from any of a plurality of directions suited to operation can be performed easily and stability of the fixed portion is also improved with the circular hole or tip portion side hole with the formation angle being in the above range.

(20) It is an operation method using any of the osteosynthesis devices according to the invention described above to fix an ulna or the ulna and free bone fragments to the intramedullary nail to perform fixation of fractured parts, the method enabling selection of an operation of performing repositioning of the fractured parts using just the intramedullary nail or an operation of performing repositioning of the fractured parts using the intramedullary nail and the endplate in combination, the method including, regardless of which operation is selected, a step of using circular holes of a plurality of directions and different angles provided in the intramedullary nail to pierce fixing pins or screws into portions suited to one's operation method, a step of using a long hole or a hole with a major axis and opening long in the same direction as the length direction of the intramedullary nail, that is provided in the intramedullary nail to pierce a fixing pin or screw at an arbitrary angle in the major axis direction, and further including, in a case where the operation of performing repositioning of the fractured parts using just the intramedullary nail is selected, a step of mounting the base end of the intramedullary nail inserted inside the ulna to be in an orientation aligned along the shape of the olecranon portion.
(21) It is an operation method using the osteosynthesis device according to the invention described above having a long hole or a hole with a major axis and opening long in the same direction as the length direction of the intramedullary nail, provided in the intramedullary nail, the intramedullary nail being a cylindrical body having a rectilinear hole extending from the base end to the tip end or having a rectilinear insertion hole extending from the base end of the intramedullary nail to the interior of the long hole that is positioned at a tip portion side or the interior of the hole with the major axis and opening long in the same direction as the length direction of the intramedullary nail, and where a fixing pin or screw is pierced at an angle orthogonal to the intramedullary nail into a bone fragment of the ulna positioned further to the base end side than a fractured or cracked portion through the long hole or the hole with the major axis and opening long in the same direction as the length direction of the intramedullary nail, provided in the intramedullary nail, to fix the intramedullary nail to the ulna and a rod-like object is inserted and pressed from the base end side of the intramedullary nail into the interior of the rectilinear hole or insertion hole to slide the fixed fixing pin or screw in the major axis direction of the same hole to move the ulna at the side fixed by the fixing pin or screw to adjust an interval between fractured portions.

In the present specification and in the claims of the present application, any material may be adopted as the material forming the respective components of the osteosynthesis device as long as, from the standpoint of a medical device used inside a human body, the material enables safety to be secured with respect to a human body and has the strength capable of withstanding use. To give specific examples, materials having biocompatibility are recommended, and although the use of titanium or a titanium alloy, which has excellent mechanical properties (light weight and high strength) and corrosion resistance, is preferable, the material is not restricted thereto and a high-strength ceramic, stainless steel or other metal material, or a plastic may be adopted.

Also, in a case where a plastic is to be used as the material, one having a bioabsorbable property is preferable in that the material is absorbed into the body after the elapse of a fixed period of time and a removal operation after complete recovery is made unnecessary, and although a polylactic acid, such as poly-L-lactic acid (PLLA), which is a bioabsorbable polymer, can be cited as an example, the material is not restricted thereto and a known bioabsorbable material, such as polyglycolic acid, etc., may be used.

As the “fixing pin or screw” as referred to in the present specification and in the claims of the present application, just one type of either fixing pin or screw may be selected and used or both a fixing pin and screw may be used. A tapping screw or other screw having threads on a shaft portion can be cited as an example of a screw, a nail or other pin without threads on a shaft portion can be cited as an example of a fixing pin, and besides such a fixing pin or screw, the fixing pin or screw may be any other known member providing the same actions and effects regardless of having or not having a screw head.

The shaft length of a fixing pin or screw may be set or selected as suited in accordance with the thickness of the bone of the treated portion and, for example, if an operation is to be performed using the intramedullary nail having the long hole near the coronoid process for a compound fracture of an olecranon portion, it is preferable to use a fixing pin or screw having a shaft length that reaches a tip end portion of the coronoid process.

Also, as a fixing pin or screw such that “a head portion or a base end of a fixing pin or screw is formed so as not to protrude further than a circumferential surface of the ulna,” for example, that with which the head portion formed at a base end portion thereof is of a shape that does not protrude further than an ulna circumferential surface at the part at which the fixing pin or screw is mounted in a state of being piercingly inserted up to the base end side during the operation or is of a shape that is flush with the ulna circumferential surface or is of a structure that does not have a head portion at the base end side can be cited, and one type of such a fixing pin or screw may be used to perform the operation or a plurality of types may be combined according to the circumstances of the fractured parts to perform the operation. If the fixing pin or screw is of a structure that does not have a head portion at the base end side, it is preferable for the fixing pin or screw to be that with which a + or − groove, a hole for hexagonal wrench, or a star-shaped hole, such as a hexalobular hole, etc., is formed in the base end surface (so-called setscrew or headless screw).

Expressions related to the respective portions of the intramedullary nail in the present specification and in the claims of the present application are based on the state where the intramedullary nail is inserted in the ulna, and in the state where the intramedullary nail is inserted in the ulna, a part positioned at an olecranon side is expressed as a “base end,” an end surface of the intramedullary nail positioned at the base end side is expressed as a “base end surface,” and a portion near the base end of the intramedullary nail is expressed as a “base end portion” or “base portion.” On the other hand, in the state where the intramedullary nail is inserted in the ulna, a part positioned at a wrist side is expressed as a “tip end” and a portion near the tip end of the intramedullary nail is expressed as a “tip end portion” or “tip portion.”

Although the part at which the “long hole” in the present specification and in the claims of the present application is provided is not restricted in particular as long as it is “a part somewhere between a center and a base end in a length direction of the intramedullary nail,” among such parts, it is more preferable for the long hole to be positioned near the coronoid process of the ulna because in this case, an operation of piercing a fixing pin or screw into the coronoid process of the ulna through the long hole is readily accommodated as well.

In addition, it is more preferable for at least one of the long holes that are provided to be positioned further to the base end side than a fractured or cracked portion because in this case, an operation method of adjusting an interval between fractured portions by moving the ulna at the side fixed by a fixing pin or screw pierced into the long hole is readily accommodated.

### Advantageous Effects of Invention

The osteosynthesis device according to the present invention has circular holes, base portion side holes, and tip portion side holes of a plurality of directions and different angles provided in the intramedullary nail and therefore is increased in the options of the insertion position or insertion direction of a fixing pin or screw despite being a single device, is thus capable of accommodating various modes of operation by the operator in accordance with the circumstances of fractured portions, and is improved in reliability of fixation of the fractured portions.

With the osteosynthesis device having the long hole or hole with the major axis and opening long in the same direction as the length direction of the intramedullary nail, the hole enables the insertion angle to be set arbitrarily and therefore the device, despite being a single device, is high in the degree of freedom of the insertion position or insertion direction of a fixing pin or screw, is thus capable of accommodating various modes of operation by the operator in accordance with the circumstances of fractured portions, and is improved in reliability of fixation of the fractured portions.

Further, with the osteosynthesis device having the end plate mounted on the outer surface of the elbow side of the ulna and where the end plate has the penetrating hole, through which is inserted the threaded screw that is inserted and screwed on from the base end of the intramedullary nail and along the length direction of the intramedullary nail, and the threaded screw is inserted and screwed on through the end plate, the operator who has selected the use of the intramedullary nail and the end plate can put the end plate in contact with the outer surface of the elbow side of the ulna and screw the end plate onto the intramedullary nail by means of the threaded screw inserted through the penetrating hole.

With the osteosynthesis device having the endplate mounted on the outer surface of the elbow side of the ulna and the threaded screw by which the end plate is screwed onto the threaded hole in the base end surface of the intramedullary nail and where the end plate has the base plate with one or more penetrating holes formed in the thickness direction and the plurality of projections projecting from the base plate toward the ulna side and contacting the outer surface of the olecranon side of the ulna, the surface of the base plate at the side opposite the ulna is formed to have no projections or to be smooth, one of either or both of the base plate and the projections of the end plate has or have flexibility or elasticity, the end plate is arranged to press the olecranon toward the tip portion side of the ulna by means of the elastic force of the base plate or the projections, and the threaded screw is used by being inserted in any one of the penetrating holes formed in the endplate, when the operator, who has selected the use of the intramedullary nail and the end plate, makes the respective projections of the end plate contact the outer surface of the elbow side of the ulna and screws the end plate onto the intramedullary nail by means of the threaded screw, the end plate, by virtue of the elastic force that it provides, presses the olecranon of the ulna toward the tip portion side of the ulna. The use of the threaded screw facilitates the adjustment of the force by which the end plate presses the olecranon of the ulna in the mounting process or adjustment in forming an interval (gap) between fractured portions. Also, the base plate front surface of the mounted end plate has no projections or is smooth and therefore the discomfort felt by a patient at the corresponding location after operation, the possibility of occurrence of a pressure sore, and the influence on tendons, etc., in the vicinity are alleviated.

With the osteosynthesis device where the shape of the base end surface of the intramedullary nail is formed to a shape conforming to the olecranon shape that includes a curved shape or formed to an obliquely-cut planar or curved shape, even if the operator selects an operation method using the intramedullary nail alone, the base end of the inserted intramedullary nail does not protrude from the olecranon side of the ulna or is unlikely to form a protrusion. Further, with the osteosynthesis device where the shape of the base end surface of the intramedullary nail is obliquely cut to the shape that is a planar shape cut in an angular range from 5° to 30°, the base end of the inserted intramedullary nail is more unlikely to protrude from the olecranon side of the ulna or is more unlikely to form a protrusion.

With the osteosynthesis device where at least two of the base portion side holes are positioned near the coronoid process of the ulna, the hole, among the holes positioned near the coronoid process, that is close to the base end portion is inclined from the base end direction to the tip end direction of the intramedullary nail so that the hole is directed in the tip end direction of the coronoid process, and the hole that is close to the tip end portion is inclined from the tip end direction to the base end direction of the intramedullary nail so that the hole is directed in the tip end direction of the coronoid process, a tip portion of a fixing pin or screw that is pierced through each base portion side hole inclined in the direction mentioned above is guided toward the tip end direction of the coronoid process, and therefore various modes of operation by the operator can be accommodated in accordance with the circumstances of fractured portions and the reliability of fixation of the fractured portions is improved.

With the osteosynthesis device where at least one of the holes with a major axis and opening long in the same direction as the length direction of the intramedullary nail is positioned further to the base end side than a fractured or cracked portion, the fractured or cracked portion is fixed by a fixing pin or screw that is pierced from the base end side toward the tip end side through the hole.

The osteosynthesis device where two holes positioned near the coronoid process are holes with a major axis opening long in the same direction as the length direction of the intramedullary nail enables, in regard to a fixing pin or screw used in each of the holes, the fixing pin or screw to be pierced at any angle with respect to the major axis direction of the hole that is the length direction of the intramedullary nail and is especially used favorably when the fixing pin or screw is to be pierced into the coronoid process of the ulna.

With the osteosynthesis device where the fixing pin or screw used in the hole, positioned near the coronoid process and through which the fixing pin or screw is inserted, has the shaft length reaching the base end or the tip end portion of the coronoid process of the ulna through the hole and capable of fastening together respective bone fragments that are separated or have a crack formed thereamong, the fixation of the fractured portions is fortified and the range of selection of operation method is broadened by the fixing pin or screw, which is pierced through the long hole or other hole positioned near the coronoid process, reaching the base end or the tip end portion of the coronoid process and further being capable of fastening together the respective bone fragments that are separated or have a crack formed thereamong.

With the osteosynthesis device where the intramedullary nail is a cylindrical body having a rectilinear hole extending from the base end to the tip end or has a rectilinear insertion hole extending from the base end of the intramedullary nail to the interior of a long hole positioned at the tip portion side or the interior of a hole with a major axis and opening long in the same direction as the length direction of the intramedullary nail, the operator can select an operation method where a fixing pin or screw is pierced into the long hole or hole with the major axis and opening long in the same direction as the length direction of the intramedullary nail, of the intramedullary nail, to fix the intramedullary nail to the ulna and a rod-like object is inserted from the base end of the intramedullary nail into the interior of the hole or insertion hole to slide the fixed fixing pin or screw in the major axis direction of the same hole to move the fixed ulna to adjust the interval between fractured portions.

With the osteosynthesis device where a female thread is provided on the inner circumferential surface of a hole, which is formed in the intramedullary nail and through which a fixing pin or screw is inserted, or the inner circumferential surface of a hole, which is formed in the end plate and through which a fixing pin or screw is inserted, when an operation using a screw is performed, the screw will be in threaded engagement with the female thread on the hole wall of each circular hole of the intramedullary nail after the operation and will practically not fall out naturally.

With the osteosynthesis device where the head portion or the base end of a fixing pin or screw is formed so as not to protrude further than an insertion portion in a circumferential surface of the ulna, the base end portion of the pierced fixing pin or screw does not project or project greatly further than the circumferential surface of the ulna and therefore the discomfort felt by a patient at the corresponding pierced location after operation, the possibility of occurrence of a pressure sore, and the influence on tendons, etc., in the vicinity are alleviated.

With the osteosynthesis device where at least the circular hole or the tip portion side hole that is positioned most distally is such that the formation angle of the circular hole, with the vertical direction of the intramedullary nail diameter being 0°, is within the range from 10° to 35°, the process of piercing a fixing pin or screw from any of a plurality of directions suited to operation can be performed easily and stability of the fixed portion is also improved because the formation angle of the circular hole or tip portion side hole is within the above range.

With the osteosynthesis device where the endplate further has one or more penetrating holes formed in the thickness direction of the base plate of the endplate and the plurality of projections projecting from the base plate toward the ulna side and contacting an outer surface of the olecranon side of the ulna, and each added penetrating hole is a hole through which is inserted a fixing pin or screw that is used together with the projections to fix the intramedullary nail near the elbow, the operator can make the respective projections of the end plate contact the outer surface of the elbow side of the ulna and screw the end plate onto the intramedullary nail by means of the threaded screw. Further, the operator can insert and pass the fixing pin or screw through the added penetrating hole as necessary and pierce the fixing pin or screw into the olecranon of the ulna or a portion in the vicinity thereof to fortify the fixation of the fractured parts near the elbow.

With the osteosynthesis device where the endplate further has one or more penetrating holes formed in the thickness direction of the base plate and each added penetrating hole is a hole through which is inserted a fixing pin or screw that is used to fix the intramedullary nail near the elbow, the operator can insert and pass the fixing pin or screw through the added penetrating hole as necessary and pierce the fixing pin or screw into the olecranon of the ulna or a portion in the vicinity thereof to fortify the fixation of the fractured parts near the elbow.

With the osteosynthesis device where one of either or both of the base plate and the projections of the end plate has or have flexibility or elasticity, the endplate is arranged to press the olecranon toward a tip portion side of the ulna by means of the elastic force of the base plate or the projections, and the surface of the base plate at the side opposite the ulna is formed to have no projections or to be smooth, when the operator makes the respective projections of the end plate contact the outer surface of the elbow side of the ulna and screws the end plate onto the intramedullary nail by means of the threaded screw, the end plate, by virtue of the elastic force that it provides, presses the olecranon of the ulna toward the tip portion side of the ulna. The use of the threaded screw facilitates adjustment of the force by which the end plate presses the olecranon of the ulna in the mounting process or adjustment in forming an interval (gap) between fractured portions. The base plate front surface of the mounted end plate has no projections or is smooth and therefore the discomfort felt by a patient at the corresponding location after operation, the possibility of occurrence of a pressure sore, and the influence on tendons, etc., in the vicinity are alleviated.

With the osteosynthesis device where the endplate is arranged with the peripheral edge portion of each of all or a portion of the penetrating holes formed in the base plate being recessed further than the base plate front surface so that the head portion of an installed threaded screw, fixing pin, or screw fits within the recess of the peripheral edge portion and the top surface of the head portion does not protrude above the base plate front surface or the base plate front surface is flush with the head portion, the head portion top surface of the fixing pin or screw does not protrude from the base plate front surface of the end plate or the base plate front surface is flush with the head portion so that the base plate front surface of the end plate has no projections or is smooth and therefore the discomfort felt by a patient at the corresponding location after operation, the possibility of occurrence of a pressure sore, and the influence on tendons, etc., in the vicinity are alleviated.

With the osteosynthesis device where the shape of the base end surface of the intramedullary nail is formed to a shape conforming to the olecranon shape that includes a curved shape or formed to an obliquely-cut planar or curved shape, even if the operator selects an operation method using the intramedullary nail alone (without using the end plate), the base end of the inserted intramedullary nail, by virtue of the above-described shape, etc., does not protrude from the olecranon side of the ulna or is unlikely to form a protrusion.

### BRIEF DESCRIPTION OF DRAWINGS

FIG. 1 is a perspective explanatory view of an osteosynthesis device D1 that is a combination of an intramedullary nail and an end plate 2a.

FIG. 2 shows a perspective explanatory view (FIG. 2(a)), a plan view (FIG. 2(b)), and a sectional view of the A-A portion (FIG. 2(c)) of the end plate 2a.

FIG. 3 shows perspective explanatory views (FIGS. 3(a) and 3(b)), a plan view (FIG. 3(c)), and a sectional view of the B-B portion (FIG. 3 (d)) of an end plate 2b.

FIG. 4 shows a plan view (FIG. 4(a)), a bottom view (FIG. 4(b)), a side view (FIG. 4(c)), a front surface side perspective view (FIG. 4(d)), a bottom surface side perspective view (FIG. 4(e)), and a sectional view of the C-C portion (FIG. 4(f)) of an end plate 2c and a sectional explanatory view of an operation of the end plate 2c combined with a threaded screw N2 (FIG. 4(g)).

FIG. 5 shows a plan view (FIG. 5(a)), a bottom view (FIG. 5(b)), a front view (FIG. 5(c)), a rear view (FIG. 5(d)), a right side view (FIG. 5(e)), a front surface side perspective view (FIG. 5(f)), a bottom surface side perspective view (FIG. 5(g)), and a sectional view of the D-D portion (FIG. 5(h)) of an end plate 2d.

FIG. 6 is a perspective explanatory view of a state where the intramedullary nail is inserted into the ulna as viewed from a distal side of the ulna.

FIG. 7 is a perspective explanatory view of the state where the intramedullary nail is inserted into the ulna as viewed from a proximal side of the ulna.

FIG. 8 is a longitudinal sectional explanatory view in a long direction of the state where the intramedullary nail is inserted into the ulna.

FIG. 9 shows a longitudinal sectional explanatory view in the long direction (FIG. 9(a)) and a transverse sectional explanatory view in the long direction (FIG. 9(b)) of an operation method using the osteosynthesis device D1.

FIG. 10 shows a longitudinal sectional explanatory view in the long direction (FIG. 10(a)) and a transverse sectional explanatory view in the long direction (FIG. 10(b)) of an operation method using an osteosynthesis device D2.

### DESCRIPTION OF EMBODIMENTS

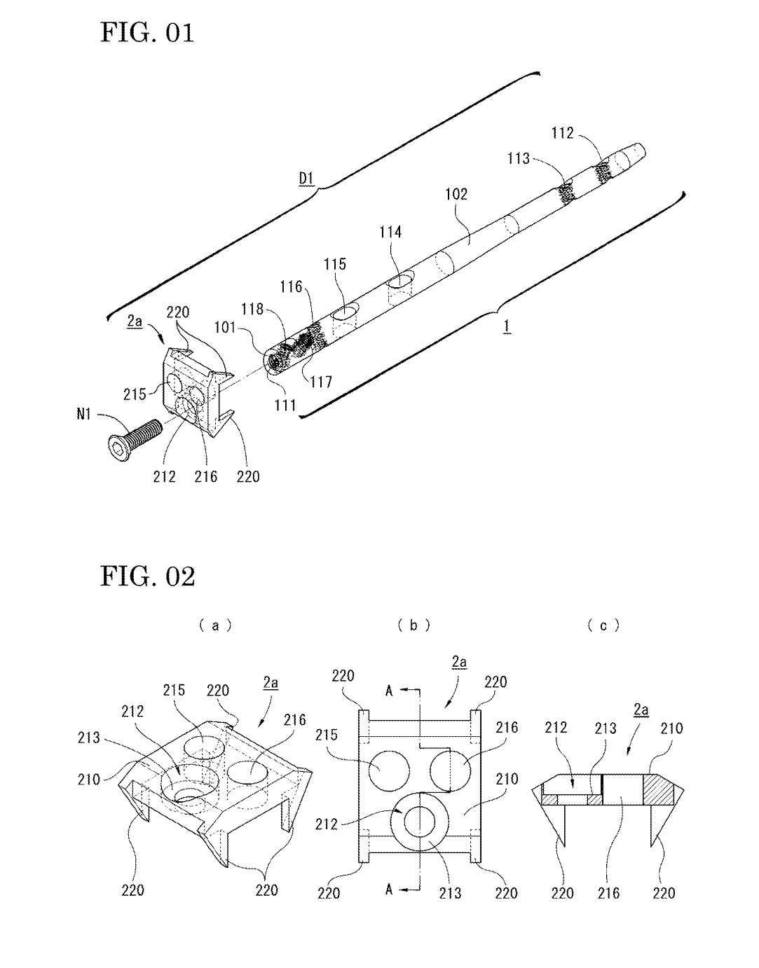

Embodiments of the present invention shall now be described in detail based on FIG. 1 to FIG. 10.

(Osteosynthesis Device D1)

An osteosynthesis device D1 is a device used, in case of fracture of an olecranon side of an ulna 3, to fix and reposition respective bone fragments. The osteosynthesis device D1 includes an intramedullary nail 1 inserted into the ulna 3, an end plate 2a mounted on an outer surface of the ulna 3 at the olecranon 31 side, and a single threaded screw N1 arranged to screw on the end plate 2a to the intramedullary nail 1 (see FIG. 1).

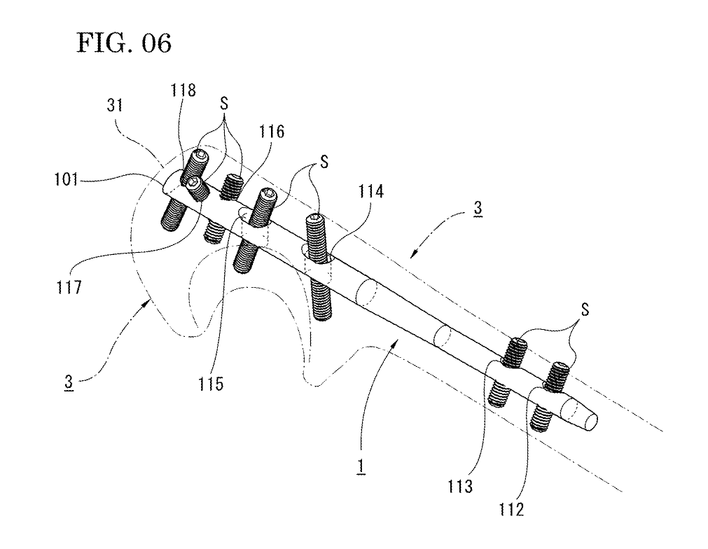

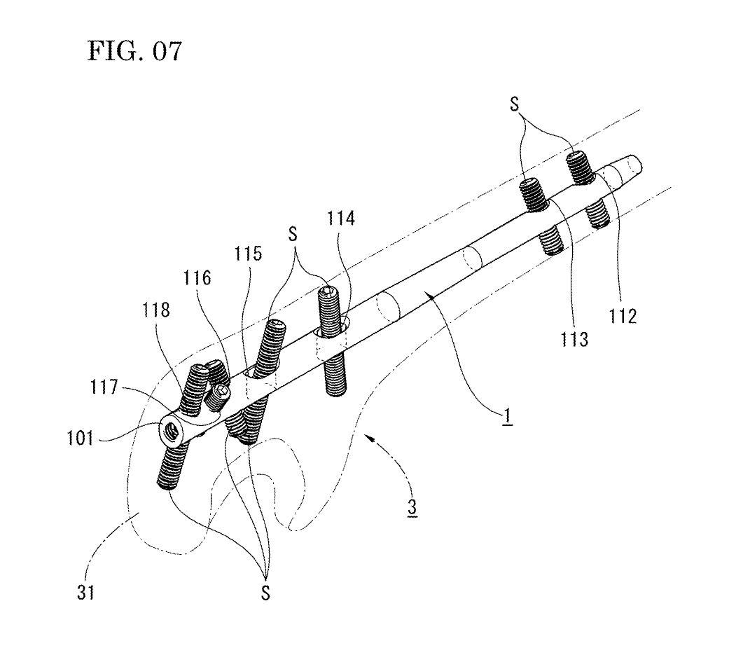

Screws S are used in combination with the osteosynthesis device D1 as fixing members for fixing, etc., the inserted intramedullary nail 1 onto the ulna 3. Each screw S is an acute threaded screw having rigidity enabling screwing into a bone and has a (headless) structure without a head portion at a base end (see FIG. 6 and FIG. 7). In the present embodiment, a titanium alloy is used as the material that forms the respective components of the osteosynthesis device.

Respective portions of the osteosynthesis device D1 shall now be described in detail.

### (Intramedullary Nail)

The intramedullary nail 1 is a round rod body having a thickness enabling entry into the marrow of the ulna, a predetermined length, and a tapering tip end portion. Although in the present embodiment, the intramedullary nail 1 has a length of approximately 100 mm, which is half the standard length of the ulna of an adult and a diameter of approximately 6 mm at a maximum portion, the length and diameter are not restricted thereto and a plurality of types of different size may be set in consideration of the physique of an operation subject. In the present embodiment, the intramedullary nail 1 is formed of a titanium alloy.

The intramedullary nail 1 includes a threaded hole 111 formed in a base end surface 101, long holes 114 and 115 formed at a base end side with respect to a central portion of a circumferential surface 102, and circular holes 112, 113, 116, 117, and 118 formed at predetermined parts between a tip portion side and the base end side of the circumferential surface 102.

Each of the long holes 114 and 115 and the circular holes 112, 113, 116, 117, and 118 penetrates through the intramedullary nail 1 in a diameter direction and all are insertion holes for the screws S. Threads are formed in a hole wall in each of the circular holes 112, 113, 116, 117, and 118.

Although in the present embodiment, the base end of the intramedullary nail 1 is formed to have an inclination angle of −15° from a highest position at the base end surface to a low position, the angle is not restricted thereto and, for example, is preferably within a range from −5° to −30° and may be changed as suited for use.

With each of the long holes 114 and 115, a major axis of each opening portion is the same in direction as a length direction of the intramedullary nail, both opening portions are same in aperture, and both long holes are orthogonal to the length direction of the intramedullary nail. With each of the long holes 114 and 115, a minor axis direction length is equal to a diameter of each circular hole and a major axis direction length is approximately twice the diameter of each circular hole. However, this scale factor must obviously be changed in accordance with a size of a crack interval and specification of the minor axis (correlated with a screw outer diameter). The scale factor is generally recommended to be within a range from 1.2 times to 5 times and preferably, a range from 1.5 times to 3 times is recommended. The present invention is not necessarily restricted to this scale factor, and the scale factor suffices to be that enabling a desired interval adjustment.

Each of the long holes 114 and 115 is arranged so that an insertion angle of a screw S may be selected freely from among any angle orthogonal to the length direction of the intramedullary nail, inclined in a direction from a tip portion direction to a base end direction of the intramedullary nail, or inclined in a direction from the base end direction to the tip portion direction of the intramedullary nail (see FIG. 6).

With each of the circular holes 112, 113, 116, and 117 among the respective circular holes, a formation direction of the hole is orthogonal to the length direction of the intramedullary nail, and with the circular hole 118, the formation direction of the hole intersects the length direction of the intramedullary nail obliquely (see FIG. 1).

Also, the circular holes 112 and 113 are equal in formation angle with respect to the diameter direction of the intramedullary nail and the circular holes 116, 117, and 118 differ respectively in formation angle with respect to the diameter direction of the intramedullary nail.

Specifically, in the present embodiment, in regard to formation directions of the holes, each of the circular holes 112, 113, 116, and 117 is formed in a direction intersecting an axial line in the length direction of the intramedullary nail at a right angle (90°) and the circular hole 118 is formed in a direction intersecting the axial line in the length direction of the intramedullary nail at 60° (see FIG. 6).

Further in the present embodiment, in regard to formation angles of the holes with respect to the diameter direction of the intramedullary nail, the circular holes 112 and 113 are inclined by 30° when compared to the long hole 114 as a reference (0°). Also, the circular hole 116 is inclined by 30° and the circular hole 117 is inclined by −30°.

The formation directions of the circular holes with respect to the axial line in the length direction of the intramedullary nail are not restricted to the numerical values given above and, for example, the angle of a circular hole provided to be inclined with respect to the length direction of the intramedullary nail may be changed as suited as long as it is within a range, for example, of 15° to 45°.

Also, the formation angles of the holes with respect to the diameter direction of the intramedullary nail are not restricted to the numerical values given above and, for example, the formation angle of a hole with respect to the diameter direction of the intramedullary nail may be changed as suited, and the formation angle, when a diameter line in a vertical direction is deemed to be 0°, is preferably in a range from 0° to 45° (or, in other words, from −45° to 45° with 0° in between) and more preferably in a range from 10° to 35° (or, in other words, from −10° to −35° and from 10° to 35° with 0° in between).

By setting each angle to be within the above range, the intramedullary nail is improved in stability within the medullary cavity, tensioning of skin (discomfort) during flexing of the elbow is reduced, the possibility of cartilage damage is reduced, and insertion of a longer screw is enabled to contribute to stability of the intramedullary nail.

(End Plate 2a)

FIGS. 2(a) to 2(c) shall now be referenced. The endplate 2a has a base plate 210 of rectangular shape in plan view and projections 220 and, in particular, the base plate 210 is formed to have flexibility or elasticity. In the present embodiment, the end plate 2a is formed of a titanium alloy.

Although the end plate 2a in the present embodiment has dimensions of approximately 12 mm in a long direction (vertical direction in FIG. 2(b)) of the base plate 210 and approximately 9 mm in a short direction (lateral direction in FIG. 2(b)), the dimensions are not restricted thereto and may be set as suited.

The base plate 210 has formed therein insertion holes 212, 215, and 216 penetrating through in a thickness direction and, with the exception of a vicinity of the insertion hole 212, a front surface (the surface at the side opposite the ulna 3 in a mounted state; hereinafter this shall be used with the same meaning) of the base plate 210 is formed to be smooth. The insertion hole 212 is a hole for insertion of the threaded screw N1 and the insertion holes 215 and 216 are holes for insertion of screws S.

The (total of four) projections 220 project sharply, one each from each corner portion of a bottom surface (the surface positioned at the ulna 3 side in a mounted state; hereinafter this shall be used with the same meaning) of the base plate 210, and each is provided with a length such that it can contact the outer surface of the ulna 3 at the olecranon 31 side or can be held across a triceps brachii without directly contacting the olecranon.

The insertion hole 212 has a shape that is cylindrically recessed from the front surface of the base plate 210, has a bottom surface, and has a hole (symbol omitted) of diameter substantially equal to the diameter of the threaded screw N1 formed at a center of the bottom surface (see FIGS. 2(a) to 2(c)). The recess is slightly larger in diameter than the diameter of a screw head of the threaded screw N1. In the present embodiment, a depth of a recess (from an opening edge portion of the insertion hole 212 to an upper surface of a bottom surface portion 213) is approximately equal to a height of the screw head of the threaded screw N1 so that when the threaded screw N1 is inserted and mounted deeply into the insertion hole 212, a lower surface side of the screw head (a boundary between the screw head and shaft; hereinafter referred to as the “neck”) of the threaded screw N1 contacts and presses the bottom surface portion 213 and a screw head top surface of the threaded screw N1 is flush with the front surface of the base plate 210.

(Actions)

Actions of the osteosynthesis device D1 and an operation method using the osteosynthesis device D1 shall now be described.

(1) After cutting the skin at an elbow portion, a hole is drilled in the olecranon 31 of the fractured ulna 3 and the intramedullary nail 1 is inserted into the marrow of the ulna 3 from the drilled part. At this time, the respective bone fragments (with the exception of small bone fragments that are free) of the fractured ulna are returned to the original positions and the intramedullary nail 1 is inserted up to its base end so as to pass through the respective bone fragments that have been repositioned and the direction of inclination of the base end surface 101 is adjusted so that the base end of the intramedullary nail 1 is set along the shape of an outer surface of the olecranon 31 or at least so that no protrusion is formed on the olecranon 31 outer surface.
(2) The positions of the circular holes 112 and 113 at the tip portion side of the intramedullary nail 1 that has been inserted into the ulna 3 are specified, the skin is cut at each corresponding portion, and screws S are screwingly inserted into the respective holes from ulna 3 side portions (the parts at which the specified circular holes 112 and 113 are positioned) and further made to project from the opposite sides of the holes to fix the tip portion of the intramedullary nail 1 to the ulna 3. At this time, each screw S may penetrate through to the ulna 3 side portion positioned at the side opposite the part of insertion.
(3) If an operator judges that further, additional fixing is required between the intramedullary nail and the ulna, the operator selects, from among the long holes 114 and 115 and the circular holes 116, 117, and 118 of the intramedullary nail 1, a hole or holes opening to a part or parts suited for the operator's operation method. The position of each of the selected one or plurality of long holes or circular holes is specified, the skin is cut at each corresponding portion (if operation is enabled from a part that has been cut already, additional cutting is not required), and a screw S is screwingly inserted into each selected long hole or circular hole from an ulna 3 side portion (the part at which the specified long hole or circular hole is positioned) to fix a central portion of the intramedullary nail 1 to the ulna 3 while keeping a tip end of the screw S from penetrating through the ulna 3 side portion positioned at the opposite side (that is, so that the tip end of the screw S stops in an interior of the ulna 3).

In regard to the long holes 114 and 115 and the circular holes 116, 117, and 118, the positions of the holes are near a joint and therefore if the tip end of a screw S is made to penetrate through the ulna 3 as in the case of the circular holes 112 and 113, the tip end may project to a joint surface, such as a trochlear notch, radial notch, or coronoid process, etc., and damage a muscle, tendon, blood vessel, or nerve, etc., when the joint portion moves, and the above procedure is thus performed to prevent this. However, in regard to the long hole 114, fixing upon penetration through the ulna 3 as in the case of the circular holes 112 and 113 is possible if a joint surface is not positioned on an axial line of the insertion angle of a screw S.

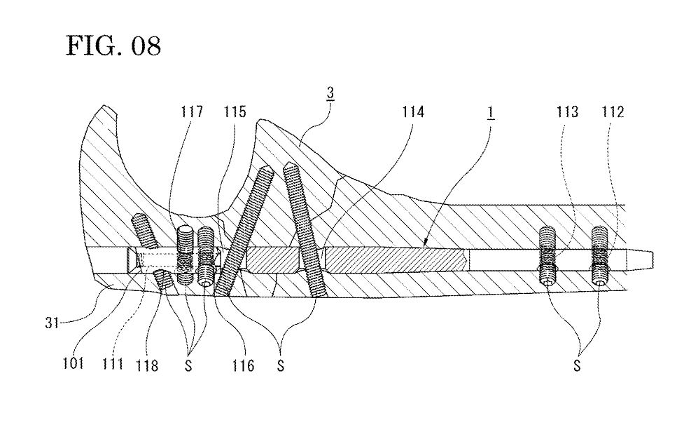

(4) If a free bone fragment is present, the free bone fragment is returned to a position of the ulna 3 where it originally should be and the intramedullary nail 1 is inserted into the ulna 3. From among the respective holes of the inserted intramedullary nail 1, a hole, with which the free bone fragment is positioned on an extension of the direction of the hole, is selected, a screw S is passed through the hole, and lastly, the free bone fragment is pierced and fixed by the screw S. By fixation of the free bone fragment to the ulna 3, the broken part will not separate again or will be unlikely to separate, and repositioning of the fractured part is thereby accomplished. A method may also be adopted where a screw S is first pierced from the free bone fragment side and then passed through the interior of the intramedullary nail 1, and lastly the tip portion of the screw S is pierced into a sound portion of the ulna 3.
(5) The operator thus uses the circular holes of plurality of directions and different angles provided in the intramedullary nail 1 to pierce screws S to parts suited for the operator's operation method to fix the fractured parts (see FIG. 6 and FIG. 7). Further, each of the long holes 114 and 115 enables a screw S to be pierced at an arbitrary angle in its major axis direction and the operator thus pierces the screw S in a direction suited to the operator's operation method to fix a fractured part or bone fragment (see FIG. 8).

After the operation, each of the screws S that has been mounted in a circular hole is substantially unlikely to fall out naturally because it is in threaded engagement with the female thread on the hole wall of the circular hole and is also unlikely to become detached or fall out at the bone or bone fragment at the pierced end because the threads on the circumferential surface of the screw S provide resistance.

[Operation Method of the Osteosynthesis Device D1 Using the End Plate 2a]

Although the operation may be completed with the working procedures (may also be referred to as “steps”) of (1) to (5) described above, further use of the endplate 2a may be selected in accordance with the state of an affected portion or a requirement of the operator's operation policy, etc.

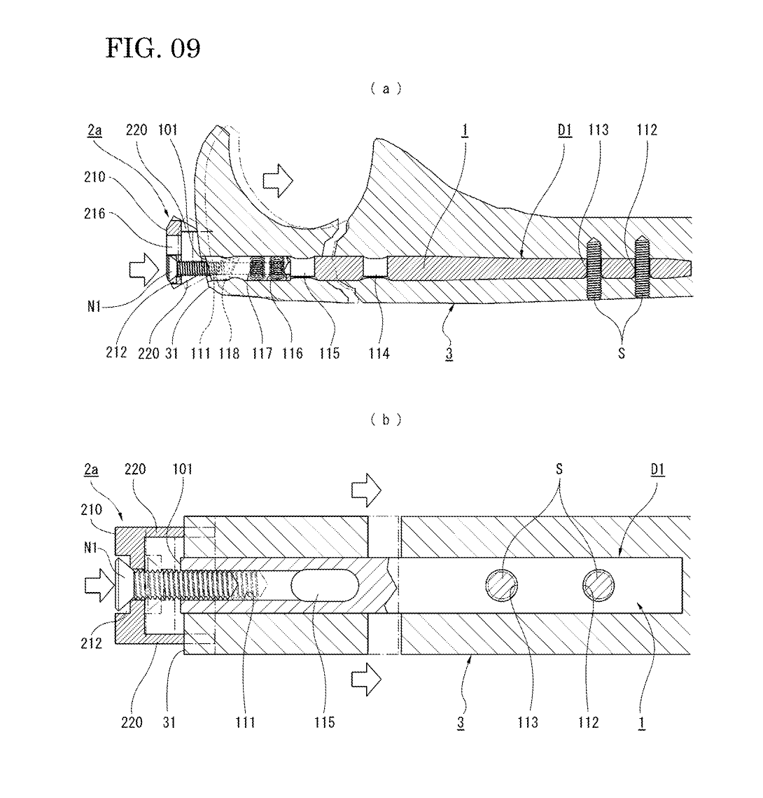

A usage state (operation method) of the osteosynthesis device D1 using the end plate 2a shall now be described with reference to FIG. 9.

Together with the steps of (1) to (5) described above, the projections 220 of the end plate 2a are mounted by being erected (may also be expressed as “pierced”) to fit the shape of the olecranon 31, and the threaded screw N1 is inserted from the insertion hole 212 and screwed into the threaded hole 111 at the base end of the intramedullary nail 1. At this time, the end plate 2a may instead be mounted so as to be held across the triceps brachii without directly contacting the olecranon 31.

By an elastic force of the end plate 2a that is generated by the tightening of the threaded screw N1, a force by which the end plate 2a presses the olecranon 31 in the ulna 3 tip portion direction is generated and the fractured portions formed in the ulna 3 are fixed in a form of pressing each other by the pressing force.

At this time, depending on the operator's operation policy or the state of the affected portion, the fractured parts may be fitted together completely as described above, or a gap may be provided between the respective bone fragments that are the fractured parts (for example, when the ulna is cut partially for repositioning and a slight gap corresponding to the width removed by the cutting is provided between the respective bone fragments in advance and subsequent regeneration is promoted to make the length of the bone return to the original length).

If the operation of providing the gap is to be performed, the tightening of the threaded screw N1 may be loosened appropriately and further, a suitable screw S may be used to adjust and achieve fixation at a position suited for the operation.

As described above, the front surface of the base plate 210 of the end plate 2a is smooth and the screw head of the threaded screw N1 does not project from the end plate 2a front surface after threaded engagement and therefore discomfort felt by a patient at the corresponding location (vicinity of the olecranon) after operation, possibility of occurrence of a pressure sore, and influence on tendons, etc., in the vicinity are alleviated.

Ordinarily with any of the operation methods, the ulna is repositioned integrally after the elapse of a fixed period of time by regeneration and joining of tissue at the joined surfaces of the respective bone fragments. The osteosynthesis device D1 may be kept fixed to the ulna 3 after complete recovery or may be removed upon observation of progress. If the osteosynthesis device D1 is made of a bioabsorbable material, it is absorbed in the body after the elapse of a fixed period of time and a removal operation after complete recovery is unnecessary.

Additionally, the operator may pierce a screw S to a portion suited for the operator's operation method through the insertion hole 215 or 216 of the end plate 2a. The end plate 2a can thereby be fixed more firmly to the olecranon 31 and the olecranon 31 or fractured portions in a vicinity thereof can be fixed not only by the intramedullary nail 1 but also from the end plate 2a side.

[End Plate 2b and Operation Method of the Osteosynthesis Device D1 Using the End Plate 2b]

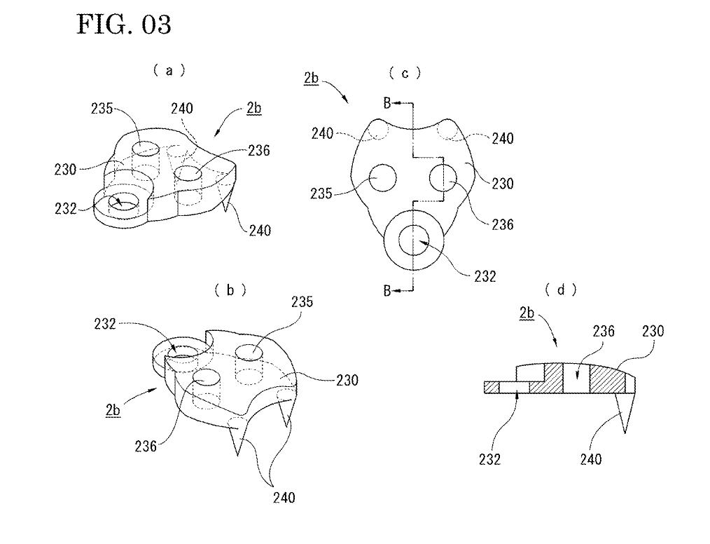

As another form of an endplate used with the osteosynthesis device D1, an end plate 2b is shown in FIGS. 3(a) to 3(d). The intramedullary nail 1 that is used in combination with the end plate 2b is the same as that described above and therefore illustration and description thereof shall be omitted.

(End Plate 2b)

The end plate 2b shown in FIG. 3 is a modification example of the end plate. The end plate 2b has a base plate 230 with a shape, which is substantially elliptical in plan view and with which one end side of its major axis is recessed arcuately toward a circle-center side (in other words, a shape arranged from different curves of different curvatures and being rounded as a whole), and sharp projections 240 (see FIGS. 3(a) to 3(d)), and has flexibility or elasticity as a whole.

The end plate 2b in the present embodiment has dimensions of approximately 13 mm in a long direction (vertical direction in FIG. 3(c)) of the base plate 230 and approximately 12.5 mm in a short direction (lateral direction in FIG. 3(c)), and has a thickness of 1.5 mm (each dimension being a dimension of a maximum portion). The dimensions of the respective portions of the end plate 2b are not restricted to the above numerical values and may be set as suited.

The base plate 230 has insertion holes 232, 235, and 236 penetrating through in a thickness direction.

The insertion holes 235 and 236 are holes for insertion of screws S and are formed opposite each other in a short direction (lateral direction of a maximum minor axis portion in FIG. 3(c)) of the end plate 2b.

The insertion hole 232 is a hole for insertion of the threaded screw N1 and is formed at another end side of a major axis (in other words, at an end portion at a side opposite the arcuate recess recessed toward the circle-center side; the lower side in FIG. 3(c)) of the end plate 2b.

With the exception of a vicinity of the insertion hole 232, the base plate 230 front surface is formed to be smooth and is inclined slightly downward from a central portion to a peripheral edge portion of the front surface of the base plate 230.

A peripheral edge of the insertion hole 232, through which the threaded screw N1 is inserted, is recessed from the front surface of the base plate 230 (in other words, thinner in thickness than portions of the base plate 230 besides the peripheral edge portion of the insertion hole 232; see FIGS. 3(a) to 3(d)) and the depth of the recess is equal to or deeper than the height of the screw head of the threaded screw N1 so that when the threaded screw N1 is mounted in the insertion hole 232, a top surface of the screw head of the threaded screw N1 is flush with the front surface of the base plate 230 or the screw head is lower.

The (total of two) projections 240 project sharply, one each from a position of the bottom surface of the base plate 230 at the side opposite the insertion hole 232 of the threaded screw N1 (at each corner portion of the arcuate recess in FIG. 3(c)), and each is provided with a length such that it can contact the outer surface at the olecranon 31 side or can be held across the triceps brachii without directly contacting the olecranon.

(Actions)

Actions of the osteosynthesis device D1 using the end plate 2b and an operation method using the osteosynthesis device D1 shall now be described. Descriptions related to the intramedullary nail 1 and descriptions of portions in common with the end plate 2a shall be omitted.

Use of the end plate 2b in addition to the fixation by the intramedullary nail 1 may be selected in accordance with the state of the affected portion or a requirement of the operator's operation policy, etc.

The projections 240 of the end plate 2b are mounted by being erected (pierced) to fit the shape of the olecranon 31, and the threaded screw N1 is inserted from the insertion hole 232 and screwed into the threaded hole 111 at the base end of the intramedullary nail 1. At this time, the end plate 2b may instead be mounted so as to be held across the triceps brachii without directly contacting the olecranon 31.

By an elastic force of the end plate 2b that is generated by the tightening of the threaded screw N1, a force by which the end plate 2b presses the olecranon 31 in the ulna 3 tip portion direction is generated and the fractured portions formed in the ulna 3 are fixed in a form of pressing each other by the pressing force.

At this time, as with the end plate 2a, either an operation of fitting the fractured parts together completely or an operation of providing a gap between the respective bone fragments may be selected by suitably adjusting the degree of tightening of the threaded screw N1, and further, a screw S may be pierced into the olecranon 31 or a fractured portion in a vicinity thereof through the insertion hole 235 or 236.

The front surface of the base plate 230 of the end plate 2b is smooth and the screw head of the threaded screw N1 does not project from the front surface of the end plate 2b after threaded engagement and therefore the discomfort at the corresponding mounting location (vicinity of the olecranon) after operation, the possibility of occurrence of a pressure sore, and the influence on tendons, etc., in the vicinity are alleviated.

(End Plate 2c)

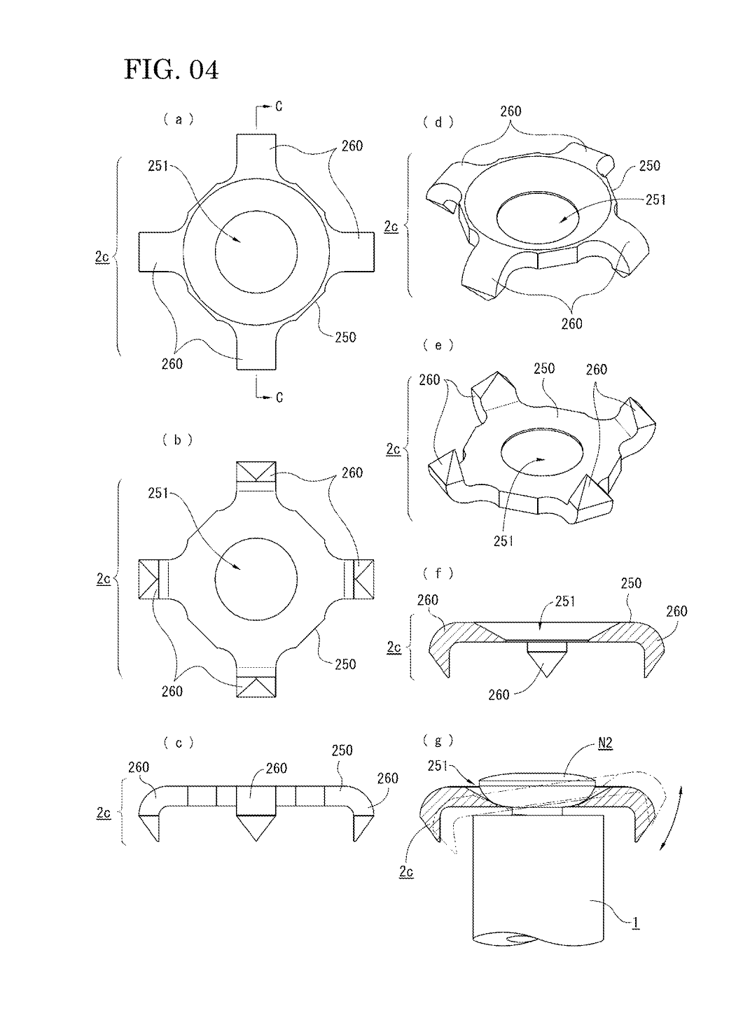

The endplate 2c shown in FIG. 4 is a modification example of the end plate. The end plate 2c has a base plate 250 with a substantially circular shape in plan view and four sharp projections 260 formed at equal intervals along a peripheral edge of the base plate (see FIGS. 4(a) to 4(f)), and has flexibility or elasticity as a whole. Small projections of substantially trapezoidal shape that become narrow in width toward a tip are formed at peripheral edge portions between the respective projections 260 of the base plate 250.

The end plate 2c in the present embodiment has diameter direction dimension of approximately 12 mm and a thickness of 2.5 mm (each dimension being a dimension of a maximum portion that includes the projections 260). The dimensions of the respective portions of the endplate 2c are not restricted to the above numerical values and may be set as suited.

The base plate 250 has an insertion hole 251 that penetrates through in a thickness direction and is a hole for insertion of a threaded screw N2. The insertion hole 251 is formed in a bowl-like shape in which an inner wall narrows gradually from a front surface side toward a bottom surface side and a hole diameter at the bottom surface side is made wider than a diameter of a shaft portion of the threaded screw N2.

With the exception of the insertion hole 251, the front surface of the base plate 250 is formed to be smooth and is arranged so that when the threaded screw N2 is mounted in the insertion hole 251, the front surface of the base plate 250 is substantially flush with a top surface of a screw head of the threaded screw N2 or the screw head is slightly higher.

Each projection 260 is sharp at a tip end portion and has a shape that projects in a horizontal direction from the peripheral edge of the base plate 250 and thereafter descends at a right angle in the direction of the bottom surface of the base plate 250 and the tip end portion is provided with a length such that it can contact the outer surface at the olecranon 31 side or can be held across the triceps brachii without directly contacting the olecranon.

Although for the endplate 2c according to the present embodiment, a side view from just one direction is shown in FIG. 4(c) and the shapes of the respective side faces (front face, rear face, right side face, and left side face) are the same as illustrated by the plan view, etc., the arrangement is not restricted thereto and an arrangement where the shapes of the side faces are changed respectively is not excluded.

(Actions)

Actions of the osteosynthesis device D1 using the end plate 2c and an operation method using the osteosynthesis device D1 shall now be described. Descriptions related to the intramedullary nail 1 and descriptions of portions in common with the end plates 2a and 2b shall be omitted.

Use of the end plate 2c in addition to the fixation by the intramedullary nail 1 may be selected in accordance with the state of the affected portion or a requirement of the operator's operation policy, etc. As in the case of the end plate 2a, the tightening of the threaded screw N2, which will be described later, may be adjusted as suited in accordance with the selection of the operation method.

With the endplate 2c, the projections 260 are mounted by being erected (pierced) to fit the shape of the olecranon 31, and the threaded screw N2 is inserted from the insertion hole 251 and screwed into the threaded hole 111 at the base end of the intramedullary nail 1. Here, the end plate 2c may instead be mounted so as to be held across the triceps brachii without directly contacting the olecranon 31.

At this time, the threaded screw N2 is tightened loosely at first and the end plate 2c is adjusted in position by being rotated or inclined around the threaded screw N2 as shown in FIG. 4(g) to position the projections 260 at parts by which fixation can be achieved most effectively in consideration of shapes of projections and recesses of the olecranon 31, positions of muscles, tendons, and ligaments, etc.

After the determination of position, the threaded screw N2 is tightened to fix the end plate 2c. If at this time, an extension in the long direction of the threaded screw N2 and the intramedullary nail 1, and the base plate 250 of the end plate 2c, are orthogonal or substantially orthogonal in side view, a neck of the threaded screw N2 is put in a position of pressing the entire side surface of the insertion hole 251 and the fractured portions of the ulna 3 are fixed by being pressed onto each other by a pressing force generated from the end plate 2c.

The end plate 2c is provided with a circular shape and therefore has an advantage of being capable of holding a central bone fragment securely during expansion and contraction of the triceps brachii.

Also, if, when the base plate 250 of the end plate 2c is attached so as to be inclined in side view with respect to the extension in the long direction of the threaded screw N2 and the intramedullary nail 1, the threaded screw N2 is tightened, the neck of the threaded screw N2 contacts a portion of the side surface of the insertion hole 251 in accordance with the inclination of the end plate 2c so that a load presses concentratingly at the part of contact and the fractured portions of the ulna 3 are fixed by being pressed onto each other by the pressing force generated from the end plate 2c.

When the end plate 2c is fixed in an inclined state, a portion of an edge portion of the threaded screw N2 protrudes slightly from the front surface of the base plate 250 and therefore the threaded screw N2 is made more rounded in the shape of the edge portion than the threaded screw N1 and an upper surface portion is made to have a roundly bulging shape to alleviate the discomfort, etc., at the corresponding mounting location (vicinity of the olecranon) after operation. In addition, the neck of the threaded screw N2 is of a shape that is more bulged arcuately in a circumferential direction in side view than the threaded screw N1 and the movable range of the end plate 2c with respect to the threaded screw N2 is thereby expanded (see FIG. 4(g)).

(End Plate 2d)

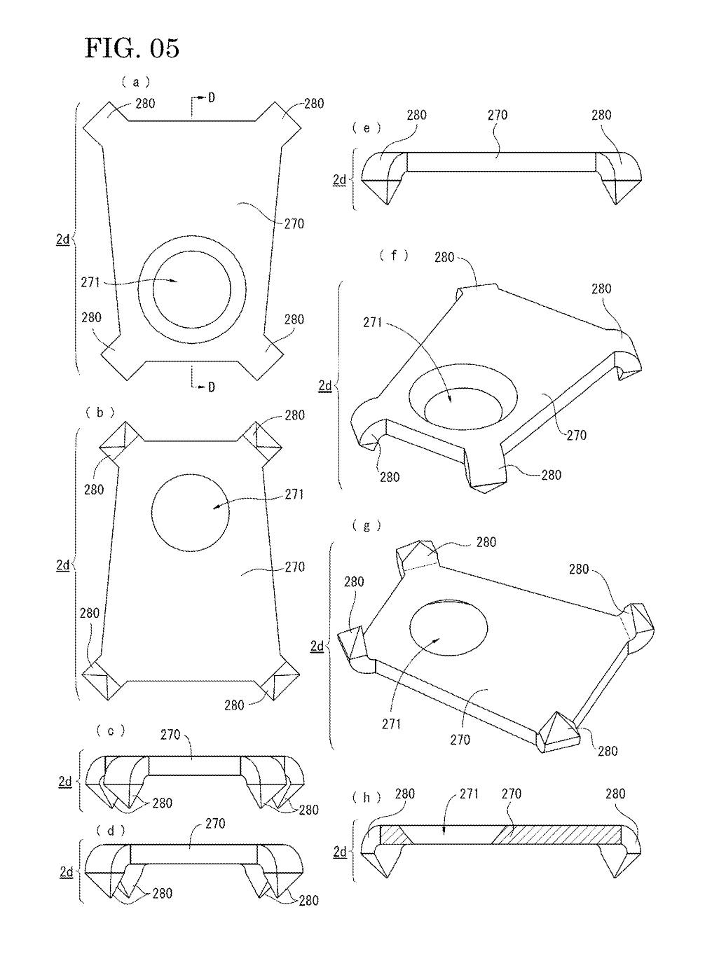

The end plate 2d shown in FIG. 5 is a modification example of the end plate. The end plate 2d has a base plate 270 with a substantially trapezoidal shape in plan view and four sharp projections 280 formed at corner portions of a peripheral edge of the base plate, and has flexibility or elasticity as a whole.

The end plate 2d in the present embodiment has dimensions of approximately 20 mm in a long direction (vertical direction in FIG. 5(a)) of the base plate 270, approximately 15 mm at a long side in a short direction (upper side in FIG. 5(a)), and approximately 10 mm at a short side in the short direction (lower side in FIG. 5(a)), and has a thickness of approximately 3 mm (each dimension being a dimension of a maximum portion that includes the projections 280). The dimensions of the respective portions of the endplate 2d are not restricted to the above numerical values and may be set as suited.

The base plate 270 has an insertion hole 271 that penetrates through in a thickness direction and is a hole for insertion of a threaded screw N2. The insertion hole 271 is formed at a center in the short direction of the base plate 270 and further toward the short side in the short direction than a center in the long direction of the base plate 270.

The insertion hole 271 is formed in a bowl-like shape in which an inner wall narrows gradually from a front surface side toward a bottom surface side and a hole diameter at the bottom surface side is made wider than a diameter of a shaft portion of the threaded screw N2.

With the exception of the insertion hole 271, the front surface of the base plate 270 is formed to be smooth and is arranged so that when the threaded screw N2 is mounted in the insertion hole 271, the front surface of the base plate 270 is substantially flush with the top surface of the screw head of the threaded screw N2 or the screw head is slightly higher.

Each projection 280 is sharp at a tip end portion and has a shape that projects from a corner portion of the base plate 270 in a horizontal direction on an extension of a center line of an interior angle of the corner portion and thereafter descends at a right angle in the direction of the bottom surface of the base plate 270 and the tip end portion is provided with a length such that it can contact the outer surface at the olecranon 31 side or can be held across the triceps brachii without directly contacting the olecranon.

Although for the endplate 2d according to the present embodiment, a left side view is omitted in FIG. 5, this is because the left side view has the same appearance as the right side view. As in end plates 2a and 2b, holes for insertion of screws S may be formed in the end plate 2d.

(Actions)

Actions of the osteosynthesis device D1 using the end plate 2d and an operation method using the osteosynthesis device D1 shall now be described. Descriptions related to the intramedullary nail 1 and descriptions of portions in common with the end plates 2a, 2b, and 2c shall be omitted.

Use of the end plate 2d in addition to the fixation by the intramedullary nail 1 may be selected in accordance with the state of the affected portion or a requirement of the operator's operation policy, etc. As in the case of the end plate 2a, the tightening of the threaded screw N2, which will be described later, may be adjusted as suited in accordance with the selection of the operation method. Also, the attachment procedure is the same as that of the end plate 2c and description thereof shall thus be omitted.

With the end plate 2d, operation is facilitated and the fitting sensation after operation is improved because the shape (the substantially trapezoidal shape in plan view) is close to the shape of the ulna, which is the subject of operation.

[Osteosynthesis Device D2 and Operation Method Using the Osteosynthesis Device D2]

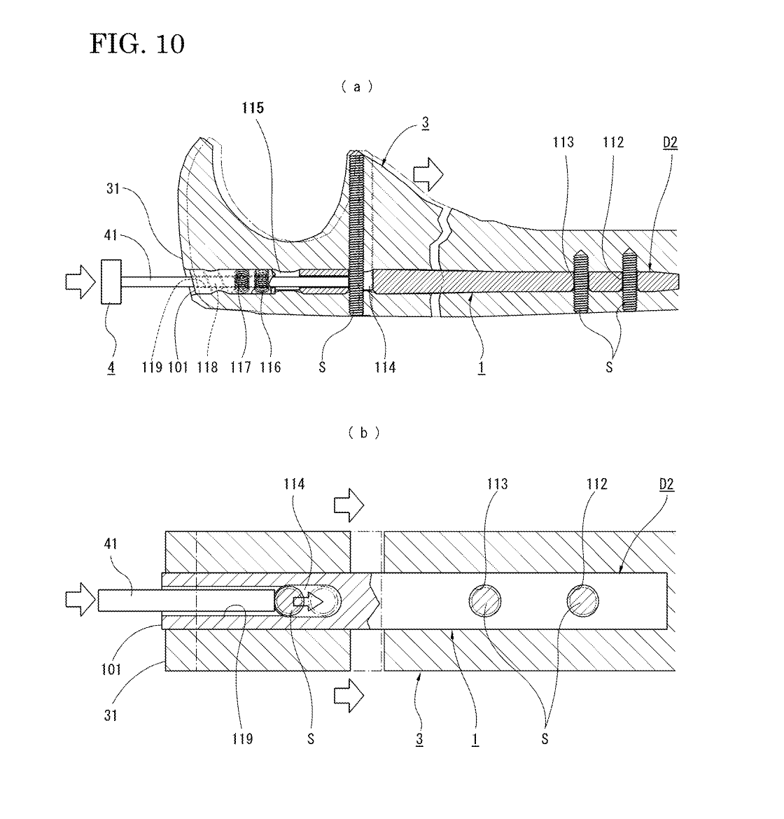

An osteosynthesis device D2 and an operation method using the osteosynthesis device D2 shall now be described with reference to FIG. 10. The osteosynthesis device D1 and the osteosynthesis device D2 differ just in a manipulating hole 119 at the base end surface 101 of the intramedullary nail 1 and description of other parts shall thus be omitted.

[Osteosynthesis Device D2]

The manipulating hole 119 is formed in the base end surface 101 of the osteosynthesis device D2. The manipulating hole 119 is formed with a length extending from the base end surface 101 to the interior of the long hole 114.

Although not shown in FIG. 10, a female thread may be formed in a hole wall of the manipulating hole 119 near the base end surface 101 and in this case, mounting of the endplate 2a or 2b is enabled.

Although with the present embodiment, the manipulating hole 119 is formed to reach the long hole 114 (the long hole at the tip end side), the arrangement is not restricted thereto and, for example, the intramedullary nail may be a cylindrical body having a rectilinear hole extending from the base end to the tip end.

(Actions)

As in the case of the osteosynthesis device D1, in the case of the osteosynthesis device D2, the skin at the elbow portion is cut, a hole is drilled in the olecranon 31 of the ulna 3, and the intramedullary nail 1 is inserted into the marrow of the ulna 3 from the drilled part so as to pass it through the respective bone fragments that have been returned to the original positions. Then, as with the osteosynthesis device D1, the direction of inclination of the base end surface 101 is adjusted so that the base end of the intramedullary nail 1 is set along the shape of the outer surface of the olecranon 31. The positions of the circular holes 112 and 113 at the tip portion side of the inserted intramedullary nail 1 are then specified, the skin is cut at each corresponding portion, and screws S are screwingly inserted into the respective holes from ulna 3 side portions and further made to project from the opposite sides of the holes to fix the tip portion of the intramedullary nail 1 to the ulna 3.

After the above step, the operator inserts a screw S into the major axis base end side of the long hole 114 of the intramedullary nail 1 so that it orthogonally crosses the long direction of the intramedullary nail 1 and makes it project from the opposite side of the hole and penetrate through the ulna 3 to fix it to the ulna 3. At this time, the base end side and the tip end side of the ulna 3 are separated near the long hole 114 (see solid line portions in FIGS. 10(a) and 10(b)).

A pressing tool 4 is then used to press the base end side of the ulna 3 toward the tip portion side to bring the fractured portions in contact. The pressing tool 4 is a T-shaped member having a holding portion (symbol omitted) and a rod-shaped guide portion 41 provided in an orthogonal direction from the holding portion.

More specifically, the guide portion 41 of the pressing tool 4 is inserted from the opening portion of the manipulating hole 119 at the base end of the intramedullary nail 1 after the fixing process and the screw S, which has been fixed at the major axis base end side of the long hole 114, is pressed toward the tip portion side of the intramedullary nail 1 by a tip end of the guide portion 41.

The screw S that is pressed slides inside the long hole 114 along the major axis direction of the long hole 114 and moves, together with the fixed ulna base end side, toward the ulna tip portion side. Consequently, the gap between the ulna base end side and the ulna tip portion side is closed and the fracture surface at the moved ulna base end side and the fracture surface at the ulna tip portion side are held in a contacting state (see alternate long and short dash line in FIGS. 10(a) and 10(b)).

If, after the step of pressing the screw S to hold the fracture surfaces in the contacting state, the operator judges that further, additional fixing is required between the intramedullary nail and the ulna, a hole or holes opening to a part or parts suited for the operator's operation method may be selected from among the long hole 115 and the circular holes 116, 117, and 118 of the intramedullary nail 1, and a screw S may be screwingly inserted and made to project from the opposite side of each selected hole to perform fixation of the fractured portions.

Although joining of the bone fragments may be achieved in the above-described state, an operation method where an endplate is mounted further to hold firmly may also be selected or, on the other hand, an operation method where a pressing force of the guide portion 41 of the pressing tool 4 is adjusted to open a gap between bone fragments to promote regeneration may also be selected.

Although with the present operation method, the pressing tool 4 is used, the member used to press the screw is not restricted in particular in shape or structure and suffices to be a member having a size and strength enabling insertion into the manipulating hole 119 and pressing of the screw S, and in an extreme case, may even be a simple metal rod, etc.

Although in the present embodiment, the member that is inserted into each long hole or each circular hole is a screw S, the member is not restricted thereto and, for example, a known fixing member, such as a fixing pin, a threaded screw, etc., may be used and further, such a fixing member preferably does not have a screw head or has a screw head (for example, of a flat plate shape, etc.) that is thin enough to be flush with or does not project greatly from the circumferential surface of an ulna to which the fixing member is mounted.

As described above, the osteosynthesis devices D1 and D2 are capable of accommodating diverse operation modes that differ according to the forms of the fractured portions and the operator.

The terms and expressions used in the present specification and claims are strictly for the purpose of description, are not restrictive whatsoever, and are not intended to exclude terms and expressions equivalent to the features described in the present specification and claims and portions thereof. Also, it is a matter of course that various modified modes are possible within the scope of the technical philosophy of the present invention.

### REFERENCE SIGN LIST

D1, D2 osteosynthesis device
1 intramedullary nail, 101 base end surface, 102 circumferential surface, 111 threaded hole, 112, 113, 116, 117, 118 circular hole, 114, 115 long hole, 119 manipulating hole
2a end plate, 210 base plate, 212 insertion hole, 213 bottom surface portion, 215, 216 insertion hole, 220 projection
2b end plate, 230 base plate, 232, 235, 236 insertion hole, 240 projection
2c end plate, 250 base plate, 251 insertion hole, 260 projection
2d end plate, 270 base plate, 271 insertion hole, 280 projection
N1, N2 threaded screw, S screw
3 ulna, 31 olecranon
4 pressing tool, 41 guide portion

1. An osteosynthesis device comprising an intramedullary nail, which, when an olecranon side of an ulna is fractured, is used by being inserted into the ulna to fix and reposition respective bone fragments,
wherein the intramedullary nail has
a threaded hole, formed in a base end surface,
a long hole, formed in a circumferential surface such that both opening portions of the long hole are the same in aperture and a fixing pin or screw can be inserted therethrough, and
at least two or more circular holes, each formed in the circumferential surface and enabling a fixing pin or screw to be inserted therethrough,
the long hole is provided at a part somewhere between a center and a base end in a length direction of the intramedullary nail, with a major axis of the long hole being the same in direction as the length direction of the intramedullary nail, and
is arranged so that an insertion angle of the fixing pin or screw inserted into the long hole may be selected freely from among any angle orthogonal to the length direction of the intramedullary nail, inclined in a direction from a tip portion direction to a base portion direction of the intramedullary nail, or inclined in a direction from the base end direction to the tip portion direction of the intramedullary nail,
the circular holes are formed at parts somewhere between a tip end and the base end in the length direction of the intramedullary nail with
formation directions of the circular holes
being orthogonal, inclined, or a combination thereof with respect to the length direction of the intramedullary nail and
being such that, with respect to a diameter direction of the intramedullary nail, the respective holes are equally overlapped with the diameter direction or a portion or all of the holes intersect the diameter direction at arbitrary angles.

2. The osteosynthesis device according to claim 1, further comprising:
an end plate mounted on an outer surface of an elbow side of the ulna and a threaded screw by which the end plate is screwed onto a threaded hole in a base end surface of the intramedullary nail;
wherein the end plate has a base plate with one or more penetrating holes formed in a thickness direction and
a plurality of projections projecting from the base plate toward the ulna side and contacting an outer surface of the olecranon side of the ulna,
a surface of the base plate at the side opposite the ulna is formed to have no projections or to be smooth,
one of either or both of the base plate and the projections of the end plate has or have flexibility or elasticity, the end plate is arranged to press the olecranon toward a tip portion side of the ulna by means of an elastic force of the base plate or the projections, and
the threaded screw is used by being inserted in any one of the penetrating holes formed in the end plate.

3. An osteosynthesis device, which, when an olecranon side of an ulna is fractured, is used to fix and reposition respective bone fragments and comprises an intramedullary nail inserted into the ulna, a threaded screw screwed on along a length direction of the intramedullary nail from a base end of the intramedullary nail, and a fixing pin or screw fixing the inserted intramedullary nail onto the ulna,
wherein the intramedullary nail has
a threaded hole formed in a base end surface,
at least one or more tip portion side holes, each being a hole provided in a circumferential surface within a range of ⅓rd the length from a tip end portion in the length direction, and
at least two or more base portion side holes, each being a hole through which the fixing pin or screw is inserted and which is provided in the circumferential surface within a range from a central portion to a base end portion in the length direction,
the holes are provided so that, with respect to a cracked portion or fractured portion of the ulna, a plurality of the base portion side holes are positioned across the cracked portion or fractured portion or a base portion side hole and a tip portion side hole are positioned so as to sandwich the cracked portion or fractured portion, and
the base portion side holes and the tip portion side holes form a positional relationship of respective holes in which insertion angles of the fixing pin or screw are combined upon being freely selected from among angles orthogonal and inclined with respect to the length direction of the intramedullary nail and angles such that, when viewed from the base end portion to the tip end portion, the respective holes are overlapped equally or intersect at arbitrary angles.

4. The osteosynthesis device according to claim 3, further comprising,
an end plate mounted on an outer surface of an elbow side of the ulna and a threaded screw by which the end plate is screwed onto a threaded hole in a base end surface of the intramedullary nail,
wherein the end plate has a base plate with one or more penetrating holes formed in a thickness direction and
a plurality of projections projecting from the base plate toward the ulna side and contacting an outer surface of the olecranon side of the ulna,
a surface of the base plate at the side opposite the ulna is formed to have no projections or to be smooth,
one of either or both of the base plate and the projections of the end plate has or have flexibility or elasticity, the end plate is arranged to press the olecranon toward a tip portion side of the ulna by means of an elastic force of the base plate or the projections, and
the threaded screw is used by being inserted in any one of the penetrating holes formed in the end plate.

5. (canceled)

6. The osteosynthesis device according to claim 2, wherein
the end plate further has one or more penetrating holes formed in the thickness direction of the base plate and each added penetrating hole is a hole through which is inserted a fixing pin or screw that is used to fix the intramedullary nail near the elbow, or
the end plate further has one or more penetrating holes formed in the thickness direction of the base plate and each added penetrating hole is a hole through which is inserted a fixing pin or screw that is used to fix the intramedullary nail near the elbow, and a female thread is provided on an inner circumferential surface of a hole, which is formed in the end plate and through which a fixing pin or screw is inserted.

7. (canceled)

8. The osteosynthesis device according to claim 4, wherein
the end plate is arranged with a peripheral edge portion of each of all or a portion of the penetrating holes formed in the base plate being recessed from the base plate front surface so that a head portion of an installed threaded screw, fixing pin, or screw fits within the recess of the peripheral edge portion and a top surface of the head portion does not protrude above the base plate front surface or the base plate front surface is flush with the head portion.

9. The osteosynthesis device according to claim 1, wherein
at least one or more of a shape of the base end surface of the intramedullary nail is formed in a shape conforming to an olecranon shape that includes a curved shape, formed in an obliquely-cut planar or curved shape, or formed in an obliquely-cut planar shape that is cut in an angular range from 5° to 30°.

10. (canceled)

11. The osteosynthesis device according to claim 3, wherein
at least two of the base portion side holes are positioned near a coronoid process of the ulna, and
among the holes positioned near the coronoid process, the hole that is close to the base end portion is inclined from the base end direction to the tip end direction of the intramedullary nail so that the hole is directed in a tip end direction of the coronoid process, and
the hole that is close to the tip end portion is inclined from the tip end direction to the base end direction of the intramedullary nail so that the hole is directed in the tip end direction of the coronoid process.

12. The osteosynthesis device according to claim 3, wherein
at least one or more of the base portion side holes is a hole with a major axis and opening long in the same direction as the length direction of the intramedullary nail, or
at least one or more of the base portion side holes is a hole with a major axis and opening long in the same direction as the length direction of the intramedullary nail, and at least one of the holes is positioned further to the base end side than a fractured or cracked portion.

13. (canceled)

14. The osteosynthesis device according to claim 3, wherein
two holes positioned near the coronoid process are holes each with a major axis and opening long in the same direction as the length direction of the intramedullary nail.

15. The osteosynthesis device according to claim 1, wherein
a fixing pin or screw used in a hole, positioned near the coronoid process and through which the fixing pin or screw is inserted, has a shaft length reaching a base end or a tip end portion of the coronoid process of the ulna through the hole and capable of fastening together respective bone fragments that are separated or have a crack formed thereamong, or
a fixing pin or screw used in a hole, positioned near the coronoid process and through which the fixing pin or screw is inserted, has a shaft length reaching a base end or a tip end portion of the coronoid process of the ulna through the hole and capable of fastening together respective bone fragments that are separated or have a crack formed thereamong, and a head portion or a base end of a fixing pin or screw is formed so as not to protrude further than an insertion portion in a circumferential surface of the ulna.

16. The osteosynthesis device according to claim 1, wherein
the intramedullary nail is a cylindrical body having a rectilinear hole extending from the base end to the tip end or has a rectilinear insertion hole extending from the base end of the intramedullary nail to the interior of a long hole positioned at a tip portion side.

17. The osteosynthesis device according to claim 9, wherein
the intramedullary nail is a cylindrical body having a rectilinear hole extending from the base end to the tip end or has a rectilinear insertion hole extending to the interior of a hole with a major axis opening long in the same direction as the length direction of the intramedullary nail.

18. The osteosynthesis device according to claim 1, wherein
a female thread is provided on an inner circumferential surface of a hole, which is formed in the intramedullary nail and through which a fixing pin or screw is inserted.

19-20. (canceled)

21. The osteosynthesis device according to claim 1, wherein
at least the circular hole that is positioned most distally is such that the formation angle of the circular hole, with a vertical direction of the intramedullary nail diameter being 0°, is within a range from 10° to 35°.

22. The osteosynthesis device according to claim 3, wherein
at least the tip portion side hole that is positioned most distally is such that the formation angle of the circular hole, with a vertical direction of the intramedullary nail diameter being 0°, is within a range from 10° to 35°.

23. The osteosynthesis device according to claim 4, wherein
the end plate further has one or more penetrating holes formed in the thickness direction of the base plate and each added penetrating hole is a hole through which is inserted a fixing pin or screw that is used to fix the intramedullary nail near the elbow, or
the end plate further has one or more penetrating holes formed in the thickness direction of the base plate and each added penetrating hole is a hole through which is inserted a fixing pin or screw that is used to fix the intramedullary nail near the elbow, and a female thread is provided on an inner circumferential surface of a hole, which is formed in the end plate and through which the fixing pin or screw is inserted.

24. The osteosynthesis device according to claim 6, wherein
the end plate is arranged with a peripheral edge portion of each of all or a portion of the penetrating holes formed in the base plate being recessed further than the base plate front surface so that a head portion of an installed threaded screw, fixing pin, or screw fits within the recess of the peripheral edge portion and a top surface of the head portion does not protrude above the base plate front surface or the base plate front surface is flush with the head portion.

25. The osteosynthesis device according to claim 3, wherein
at least one or more of a shape of the base end surface of the intramedullary nail is formed in a shape conforming to an olecranon shape that includes a curved shape, formed in an obliquely-cut planar or curved shape, or formed in an obliquely-cut planar shape that is cut in an angular range from 5° to 30°.

26. The osteosynthesis device according to claim 3, wherein
a fixing pin or screw used in a hole, positioned near the coronoid process and through which the fixing pin or screw is inserted, has a shaft length reaching a base end or a tip end portion of the coronoid process of the ulna through the hole and capable of fastening together respective bone fragments that are separated or have a crack formed thereamong, or
a fixing pin or screw used in a hole, positioned near the coronoid process and through which the fixing pin or screw is inserted, has a shaft length reaching a base end or a tip end portion of the coronoid process of the ulna through the hole and capable of fastening together respective bone fragments that are separated or have a crack formed thereamong, and a head portion or a base end of the fixing pin or screw is formed so as not to protrude further than an insertion portion in a circumferential surface of the ulna.
